(19) United States
(12) Patent Application Publication
(10) Pub. No.: US 2023/0277085 A1
(43) Pub. Date: Sep. 7, 2023

(54) SENSOR ATTACHMENT SYSTEM, RESPIRATORY GAS SENSOR, AND COMPATIBLE AIRWAY ADAPTOR

(71) Applicant: NIHON KOHDEN CORPORATION, Tokyo (JP)

(72) Inventors: Kenichiro KABUMOTO, Tokorozawa-shi (JP); Fumihiko TAKATORI, Tokorozawa-shi (JP); Yuya BABA, Tokorozawa-shi (JP)

(21) Appl. No.: 18/169,400

(22) Filed: Feb. 15, 2023

(30) Foreign Application Priority Data
Mar. 7, 2022 (JP) 2022-034701

(51) Int. Cl. A61B 5/097; A61M 16/08

(52) U.S. Cl. CPC A61B 5/097; A61M 16/085; A61M 2230/40

(57) ABSTRACT

A sensor attachment system includes: a sensor configured to detect a state of respiratory gas of a subject; and a compatible airway adaptor compatible with the sensor. The sensor includes a respiratory gas detector configured to detect the state of the respiratory gas of the subject, and a sensor-side engagement portion engageable with the compatible airway adaptor. The compatible airway adaptor includes a tube portion having a respiratory gas ventilation passage through which the respiratory gas passes, and an adapter-side engagement portion engageable with the sensor-side engagement portion. The sensor is attachable to the compatible airway adaptor in a state in which the compatible airway adaptor and the sensor do not interfere with each other, and the sensor is not attachable to an incompatible airway adaptor that is not compatible with the sensor in a state in which the incompatible airway adaptor and the sensor interfere with each other.

### CROSS-REFERENCE TO RELATED APPLICATIONS

This application is based on and claims priority under 35 USC 119 from Japanese Patent Application No. 2022-034701 filed on Mar. 7, 2022, the contents of which are incorporated herein by reference.

### TECHNICAL FIELD

The present disclosure relates to a sensor attachment system, a respiratory gas sensor, and a compatible airway adaptor.

### BACKGROUND

Japanese Patent Application Laid-Open Publication No. 2019-174152 discloses a sensor attachment system including an airway adaptor connectable to a tracheal tube of a respirator and a respiratory gas sensor attachable to the airway adaptor. By attaching the respiratory gas sensor to the airway adaptor, a state of exhaled gas of a patient (for example, concentration or partial pressure of carbon dioxide) can be detected. To improve the measurement accuracy of information measured by the respiratory gas sensor, the respiratory gas sensor is attached to the airway adaptor in a state in which the respiratory gas sensor and the airway adaptor are positioned.

In general, the respiratory gas sensor is repeatedly used, while the airway adaptor is disposable. Various types of airway adaptors are prepared according to applications, such as non-intubation airway adaptors and intubation airway adaptors. Here, an intubation airway adaptor is an airway adaptor used for invasive positive pressure ventilation in which a tracheal tube is inserted into a trachea of a patient. On the other hand, a non-intubation airway adaptor is an airway adaptor used for non-invasive positive pressure ventilation in which no tracheal tube is inserted into a trachea of a patient. In general, the respiratory gas sensor is attachable to all types of airway adapters.

When the respiratory gas sensor is compatible with both invasive positive pressure ventilation and non-invasive positive pressure ventilation, it is preferable that the respiratory gas sensor is attachable to all types of airway adapters (in particular, intubation airway adapters and non-intubation airway adapters). On the other hand, when the respiratory gas sensor is applicable only to invasive positive pressure ventilation, it may not be preferable that the respiratory gas sensor is attachable to a non-intubation airway adaptor. For example, to improve the measurement accuracy of a CO₂ concentration in respiratory gas (exhaled gas) of a patient, calibration of the CO₂ concentration of the respiratory gas sensor is generally performed before using the respiratory gas sensor. A calibration method for the CO₂ concentration may be different between invasive positive pressure ventilation and non-invasive positive pressure ventilation. When the respiratory gas sensor to which the calibration method for invasive positive pressure ventilation is applied is attached to a non-intubation airway adaptor, the respiratory gas sensor may not be able to detect the CO₂ concentration of the patient with high accuracy. As another example, a respiratory gas sensor for an adult may be assumed to be attached to an airway adaptor for a neonate. In this case, it is assumed that a tracheal tube connected to a neonatal airway adaptor is bent excessively due to the weight of the respiratory gas sensor for an adult. In this manner, there is room for consideration of a sensor attachment system in which a respiratory gas sensor is attachable or not attachable to an airway adaptor depending on a type of the airway adaptor.

### SUMMARY

A first aspect of the present disclosure relates to a sensor attachment system including: a sensor configured to detect a state of respiratory gas of a subject; and a compatible airway adaptor compatible with the sensor. The sensor includes a respiratory gas detector configured to detect the state of the respiratory gas of the subject, and a sensor-side engagement portion engageable with the compatible airway adaptor. The compatible airway adaptor includes a tube portion having a respiratory gas ventilation passage through which the respiratory gas passes, and an adapter-side engagement portion engageable with the sensor-side engagement portion. The sensor is attachable to the compatible airway adaptor in a state in which the compatible airway adaptor and the sensor do not interfere with each other, and the sensor is not attachable to an incompatible airway adaptor that is not compatible with the sensor in a state in which the incompatible airway adaptor and the sensor interfere with each other.

A second aspect of the present disclosure relates to a respiratory gas sensor. The respiratory gas sensor is configured to detect a state of respiratory gas of a subject and is attachable to a compatible airway adaptor compatible with the respiratory gas sensor. The respiratory gas sensor includes: a respiratory gas detector configured to detect the state of the respiratory gas of the subject; and a sensor-side engagement portion engageable with the compatible airway adaptor. The respiratory gas sensor is attachable to the compatible airway adaptor in a state in which the compatible airway adaptor and the respiratory gas sensor do not interfere with each other, and the respiratory gas sensor is not attachable to an incompatible airway adaptor that is not compatible with the respiratory gas sensor in a state in which the incompatible airway adaptor and the respiratory gas sensor interfere with each other.

A third aspect of the present disclosure relates to a compatible airway adaptor. The compatible airway adaptor is attachable to a respiratory gas sensor configured to detect a state of respiratory gas of a subject. The compatible airway adaptor includes: a tube portion having a respiratory gas ventilation passage through which the respiratory gas passes; and an adapter-side engagement portion configured to engage with the respiratory gas sensor. The respiratory gas sensor includes an attachment preventing portion configured to cause the respiratory gas sensor to be unattachable to an incompatible airway adaptor that is not compatible with the respiratory gas sensor, and the compatible airway adaptor further includes an accommodation portion configured to accommodate the attachment preventing portion to prevent interference with the attachment preventing portion.

### BRIEF DESCRIPTION OF DRAWINGS

Exemplary embodiment(s) of the present invention will be described in detail based on the following figures, wherein:

FIG. 1 is a perspective view of a sensor attachment system including a respiratory gas sensor and a compatible airway adaptor;

FIG. 2 is a front view of the respiratory gas sensor;

FIG. 3 is a back view of the respiratory gas sensor;

FIG. 4 is a bottom view of the respiratory gas sensor;

FIG. 5 is a perspective view of the compatible airway adaptor;

FIG. 6 is a perspective view of the respiratory gas sensor and an incompatible airway adaptor; and

FIG. 7 is a perspective view of the incompatible airway adaptor.

### DESCRIPTION OF EMBODIMENTS

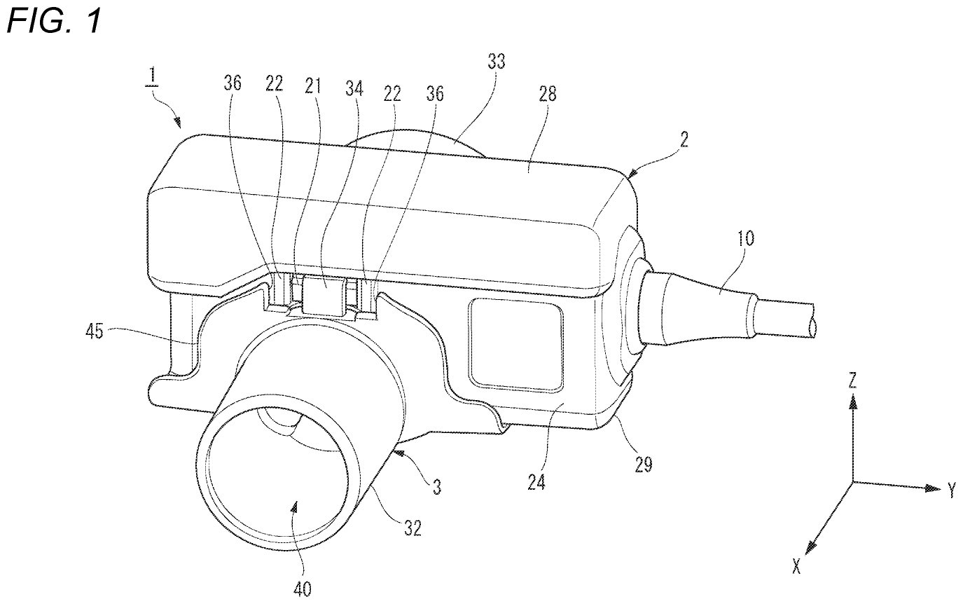

A sensor attachment system 1 according to an embodiment of the present disclosure (hereinafter, referred to as the present embodiment) will be described below with reference to the drawings. Dimensions of members illustrated in the drawings may be different from actual dimensions of the members for the sake of convenience of description. In the present embodiment, an X-axis direction, a Y-axis direction, and a Z-axis direction set for the sensor attachment system 1 illustrated in FIG. 1 will be appropriately referred to. Each of the X-axis direction, the Y-axis direction, and the Z-axis direction is perpendicular to the remaining two directions. For example, the X-axis direction is perpendicular to the Y-axis direction and the Z-axis direction.

FIG. 1 is a perspective view of the sensor attachment system 1 that can include a respiratory gas sensor 2 and a compatible airway adaptor 3. As illustrated in FIG. 1, the sensor attachment system 1 can include the respiratory gas sensor 2 and the compatible airway adaptor 3. The respiratory gas sensor 2 detects a state of respiratory gas (for example, concentration or partial pressure of carbon dioxide contained in respiratory gas) of a patient (subject). The respiratory gas sensor 2 is connected to a patient monitor (not illustrated) through a cable 10. The patient monitor obtains a vital signs signal indicating the state of the respiratory gas of the patient from the respiratory gas sensor 2, and then displays information indicating the state of the respiratory gas (exhaled gas) on a display screen.

The compatible airway adaptor 3 is an airway adaptor compatible with the respiratory gas sensor 2. That is, the respiratory gas sensor 2 is attachable to the compatible airway adaptor 3. More specifically, the respiratory gas sensor 2 is attachable to the compatible airway adaptor 3 in a state in which the compatible airway adaptor 3 and the respiratory gas sensor 2 do not interfere with each other. FIG. 1 illustrates a state in which the respiratory gas sensor 2 is attached to the compatible airway adaptor 3. The airway adaptor defines a ventilation passage through which the respiratory gas of the patient passes. When the airway adaptor is an intubation airway adaptor used for invasive positive pressure ventilation, the airway adaptor is connected to a tracheal tube inserted into a trachea of the patient. When the airway adaptor is a non-intubation airway adaptor used for non-invasive positive pressure ventilation, the airway adaptor may be attached to an oxygen mask.

### <Configurations of Respiratory Gas Sensor>

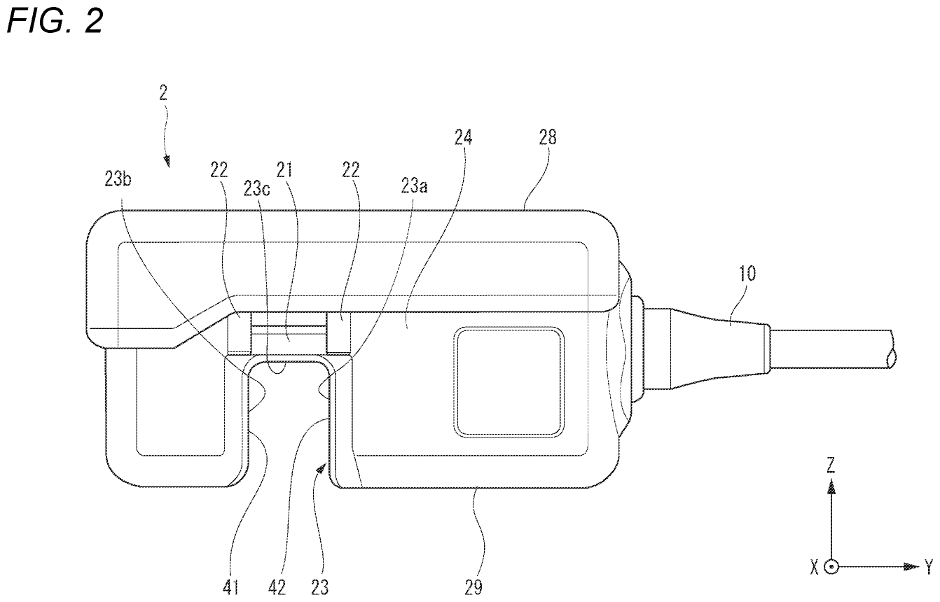

Next, configurations of the respiratory gas sensor 2 will be specifically described with reference to FIGS. 1 to 4. As illustrated in FIGS. 1 to 4, the respiratory gas sensor 2 can include a first surface 24, a second surface 27 opposite to the first surface 24, an upper surface 28, and a lower surface 29 opposite to the upper surface 28. As illustrated in FIG. 2, the respiratory gas sensor 2 is provided with a first sensor-side engagement portion 21 on the first surface 24. The first sensor-side engagement portion 21 engages with a portion of the compatible airway adaptor 3. The respiratory gas sensor 2 is provided with a pair of first attachment preventing portions 22 on the first surface 24. The pair of first attachment preventing portions 22 face each other through the first sensor-side engagement portion 21. Each of the first attachment preventing portions 22 has a columnar body protruding from the first surface 24. Each of the first attachment preventing portions 22 extends along the Z-axis direction, which is an insertion direction in which the respiratory gas sensor 2 is inserted into the compatible airway adaptor 3.

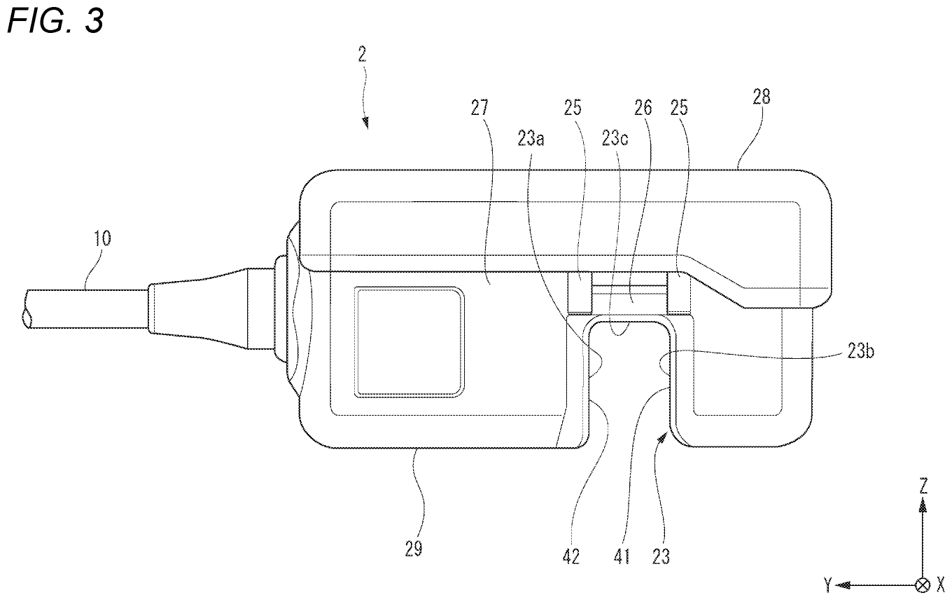

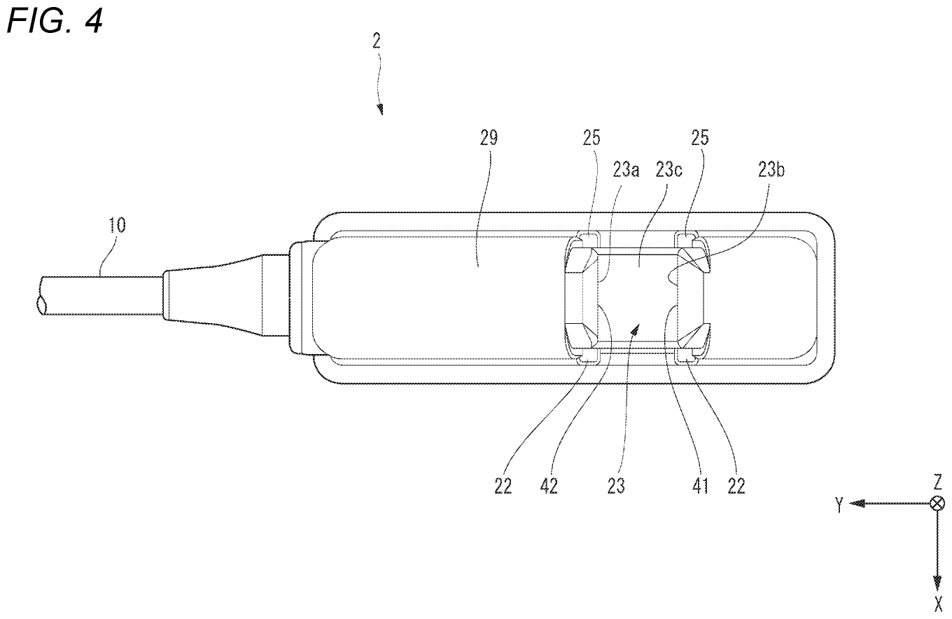

As illustrated in FIG. 3, the respiratory gas sensor 2 is provided with a second sensor-side engagement portion 26 on the second surface 27. The second sensor-side engagement portion 26 engages with a portion of the compatible airway adaptor 3. The respiratory gas sensor 2 is provided with a pair of second attachment preventing portions 25 on the second surface 27. The pair of second attachment preventing portions 25 face each other through the second sensor-side engagement portion 26. Each of the second attachment preventing portions 25 has a columnar body protruding from the second surface 27. Each of the second attachment preventing portions 25 extends along the Z-axis direction. As illustrated in FIG. 4, each of the pair of second attachment preventing portions 25 faces a corresponding one of the pair of first attachment preventing portions 22 in the X-axis direction.

As illustrated in FIGS. 2 to 4, the respiratory gas sensor 2 is provided with a fitting portion 23 on the lower surface 29. The fitting portion 23 determines a position of the respiratory gas sensor 2 relative to the compatible airway adaptor 3. A portion of the compatible airway adaptor 3 is fitted to the fitting portion 23, such that the respiratory gas sensor 2 is attached to the compatible airway adaptor 3. The fitting portion 23 is a groove penetrating a body of the respiratory gas sensor 2 in the X-axis direction. The fitting portion 23 extends between the first surface 24 and the second surface 27 in the X-axis direction, and extends between the lower surface 29 and the first sensor-side engagement portion 21 (or the second sensor-side engagement portion 26) in the Z-axis direction. In the Z-axis direction, the first sensor-side engagement portion 21 and the second sensor-side engagement portion 26 face the fitting portion 23.

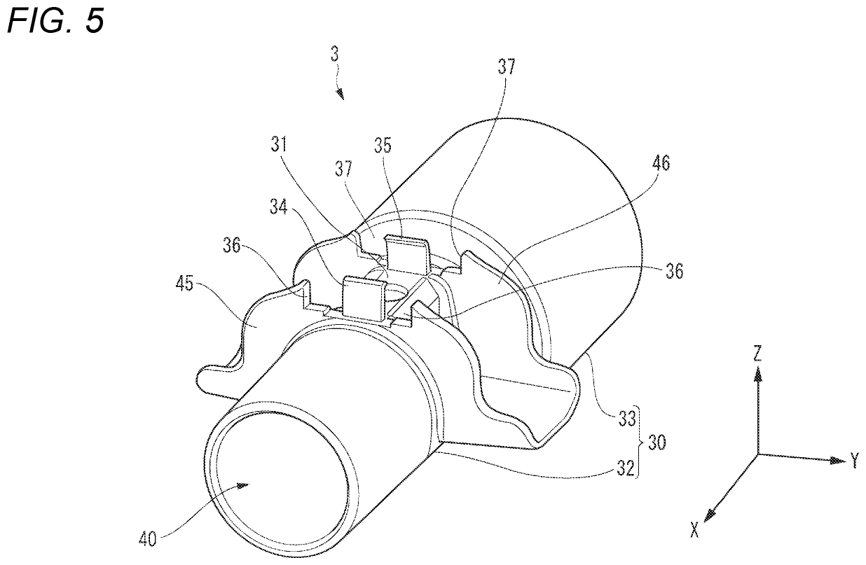

The fitting portion 23 is defined by side wall portions 23a and 23b facing each other in the Y-axis direction and a bottom portion 23c connected to a corresponding end of each of the side wall portions 23a and 23b. The side wall portion 23a is provided with a light emitting unit 42. The side wall portion 23b is provided with a light detecting unit 41. That is, the light emitting unit 42 and the light detecting unit 41 face each other through the fitting portion 23 in the Y-axis direction. As illustrated in FIG. 1, a sensor attachment portion 31 (see FIG. 5) is fitted to the fitting portion 23 in a state in which the respiratory gas sensor 2 is attached to the compatible airway adaptor 3. In this state, the light emitting unit 42 and the light detecting unit 41 face each other in the Y-axis direction through the respiratory gas passing through a respiratory gas ventilation passage 40.

The light emitting unit 42 and the light detecting unit 41 function as a respiratory gas detector that detects the state of the respiratory gas of the patient. The light emitting unit 42 can include a first light emitter that emits first infrared light and a second light emitter that emits second infrared light having a wavelength different from that of the first infrared light. The light detecting unit 41 can include a first light detector that detects the first infrared light emitted from the first light emitter and a second light detector that detects the second infrared light emitted from the second light emitter. In the present embodiment, the respiratory gas detector is not limited to the light emitting unit and the light detecting unit.

The first infrared light is absorbed by carbon dioxide contained in the respiratory gas, whereas the second infrared light is not absorbed by carbon dioxide. In this manner, an intensity of the first infrared light detected by the first light detector changes according to a concentration of carbon dioxide contained in the respiratory gas. On the other hand, a detected light intensity of the second infrared light detected by the second light detector does not change according to the concentration of carbon dioxide. Therefore, the first infrared light is used as measurement light for measuring the concentration of carbon dioxide, while the second infrared light is used as reference light. A controller (not illustrated) provided in the respiratory gas sensor 2 can generate a vital signs signal indicating the state of the respiratory gas of the patient (concentration or partial pressure of carbon dioxide contained in respiratory gas) based on a ratio between an intensity of a first measurement signal output from the first light detector and an intensity of a second measurement signal output from the second light detector.

The controller may perform calibration processing of the carbon dioxide concentration before generating the vital signs signal indicating the state of the respiratory gas of the patient. By performing the calibration processing in advance, the measurement accuracy of the carbon dioxide concentration can be improved. A method for the calibration processing of the carbon dioxide concentration may be different depending on invasive positive-pressure ventilation or non-invasive positive-pressure ventilation.

### <Configurations of Compatible Airway Adapter>

Next, configurations of the compatible airway adaptor 3 will be specifically described with reference to FIG. 5. FIG. 5 is a perspective view of the compatible airway adaptor 3. As illustrated in FIG. 5, the compatible airway adaptor 3 can include a tube portion 30, the sensor attachment portion 31, a first flange portion 45, a second flange portion 46, a first adapter-side engagement portion 34, and a second adapter-side engagement portion 35. The compatible airway adaptor 3 can further include a pair of first accommodation portions 36 and a pair of second accommodation portions 37.

The tube portion 30 can include the respiratory gas ventilation passage 40 through which the respiratory gas exhaled from the patient passes. The tube portion 30 can include a first tube portion 32 having a first outer diameter and a second tube portion 33 having a second outer diameter greater than the first outer diameter. Both of the first tube portion 32 and the second tube portion 33 have a cylindrical shape, and the respiratory gas ventilation passage 40 is implemented by a hollow region of the first tube portion 32 and a hollow region of the second tube portion 33. The first tube portion 32 is connected to an external device such as a respirator or an airbag through a tube. The second tube portion 33 is connected to a device attached to the patient, such as a tracheal tube or a mask.

The sensor attachment portion 31 determines a position of the respiratory gas sensor 2 relative to the compatible airway adaptor 3. When the respiratory gas sensor 2 is attached to the compatible airway adaptor 3, the sensor attachment portion 31 is fitted to the fitting portion 23 of the respiratory gas sensor 2. The sensor attachment portion 31 is disposed between the first tube portion 32 and the second tube portion 33 in the X-axis direction, which is a longitudinal direction of the compatible airway adaptor 3. The sensor attachment portion 31 has a hollow region. The hollow region of the sensor attachment portion 31 communicates with the hollow region of the first tube portion 32 and the hollow region of the second tube portion 33. In this manner, the respiratory gas of the patient passes through the hollow region of the second tube portion 33, and then passes through the hollow region of the sensor attachment portion 31 and the hollow region of the first tube portion 32.

The sensor attachment portion 31 is provided with a pair of glass windows (not illustrated) facing each other in the Y-axis direction. The pair of glass windows transmits infrared light. In a state in which the respiratory gas sensor 2 is attached to the compatible airway adaptor 3, one of the pair of glass windows faces the light emitting portion 42 in the Y-axis direction, and the other one of the pair of glass windows faces the light detecting unit 41 in the Y-axis direction.

The first flange portion 45 is disposed between the first tube portion 32 and the sensor attachment portion 31 in the X-axis direction. In a state in which the respiratory gas sensor 2 is attached to the compatible airway adaptor 3, the first flange portion 45 covers a portion of the first surface 24 of the respiratory gas sensor 2 in the X-axis direction. The second flange portion 46 is disposed between the second tube portion 33 and the sensor attachment portion 31 in the X-axis direction. In a state in which the respiratory gas sensor 2 is attached to the compatible airway adaptor 3, the second flange portion 46 covers a portion of the second surface 27 of the respiratory gas sensor 2 in the X-axis direction.

The first adapter-side engagement portion 34 engages with the first sensor-side engagement portion 21 of the respiratory gas sensor 2 (see FIG. 1). In particular, the first adapter-side engagement portion 34 is elastically deformed when the respiratory gas sensor 2 is attached to the compatible airway adaptor 3, such that the first adapter-side engagement portion 34 is snap-fitted to the first sensor-side engagement portion 21. The first adapter-side engagement portion 34 is disposed between the first tube portion 32 and the sensor attachment portion 31 in the X-axis direction.

The second adapter-side engagement portion 35 engages with the second sensor-side engagement portion 26 (see FIG. 3) of the respiratory gas sensor 2. In particular, the second adapter-side engagement portion 35 is elastically deformed when the respiratory gas sensor 2 is attached to the compatible airway adaptor 3, so that the second adapter-side engagement portion 35 is snap-fitted to the second sensor-side engagement portion 26. The second adapter-side engagement portion 35 is disposed between the second tube portion 33 and the sensor attachment portion 31 in the X-axis direction. The second adapter-side engagement portion 35 and the first adapter-side engagement portion 34 face each other in the X-axis direction.

The pair of first accommodation portions 36 prevent interference between the respiratory gas sensor 2 and the compatible airway adaptor 3. The pair of first accommodation portions 36 are disposed between the first tube portion 32 and the sensor attachment portion 31 in the X-axis direction, and face each other through the first adapter-side engagement portion 34 in the Y-axis direction. The pair of first accommodation portions 36 are cutout portions provided in the first flange portion 45. As illustrated in FIG. 1, each of the pair of first accommodation portions 36 accommodates a corresponding one of the pair of first attachment preventing portions 22. In particular, each of the pair of first accommodation portions 36 accommodates the corresponding first attachment preventing portion 22 to prevent interference between the corresponding first attachment preventing portion 22 and the compatible airway adaptor 3.

The pair of second accommodation portions 37 prevent interference between the respiratory gas sensor 2 and the compatible airway adaptor 3. The pair of second accommodation portions 37 are disposed between the second tube portion 33 and the sensor attachment portion 31 in the X-axis direction, and face each other through the second adapter-side engagement portion 35 in the Y-axis direction. The pair of second accommodation portions 37 are cutout portions provided in the second flange portion 46. Each of the pair of second accommodation portions 37 accommodates a corresponding one of the pair of second attachment preventing portions 25 (see FIG. 3). In particular, each of the pair of second accommodation portions 37 accommodates the corresponding second attachment preventing portion 25 to prevent interference between the corresponding second attachment preventing portion 25 and the compatible airway adaptor 3. Each of the pair of second accommodation portions 37 faces a corresponding one of the pair of first accommodation portions 36 in the X-axis direction.

In the present embodiment, since the compatible airway adaptor 3 is provided with the pair of first accommodation portions 36 and the pair of second accommodation portions 37, interference between the respiratory gas sensor 2 and the compatible airway adaptor 3 is prevented, and the compatible airway adaptor 3 is attachable to the respiratory gas sensor 2.

### <Configurations of Incompatible Airway Adapter>

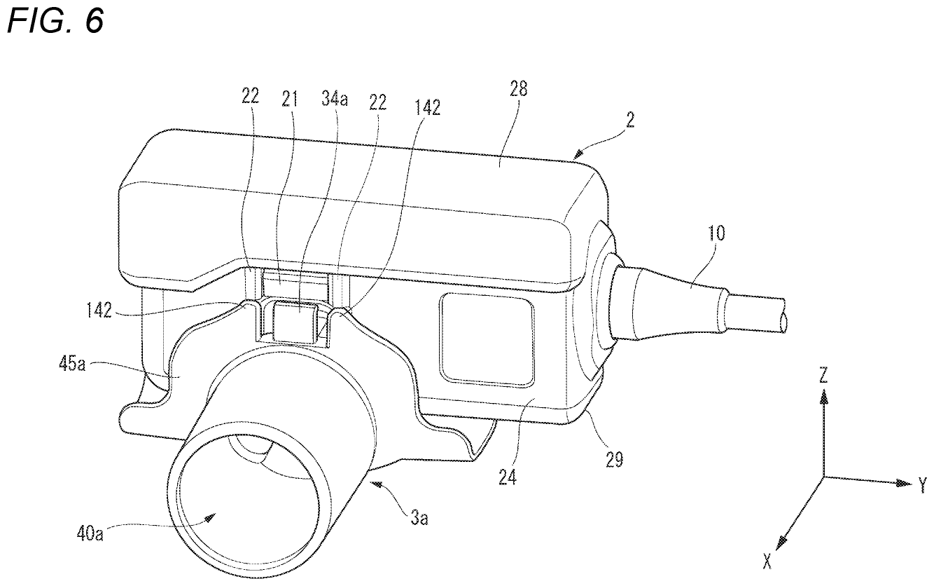

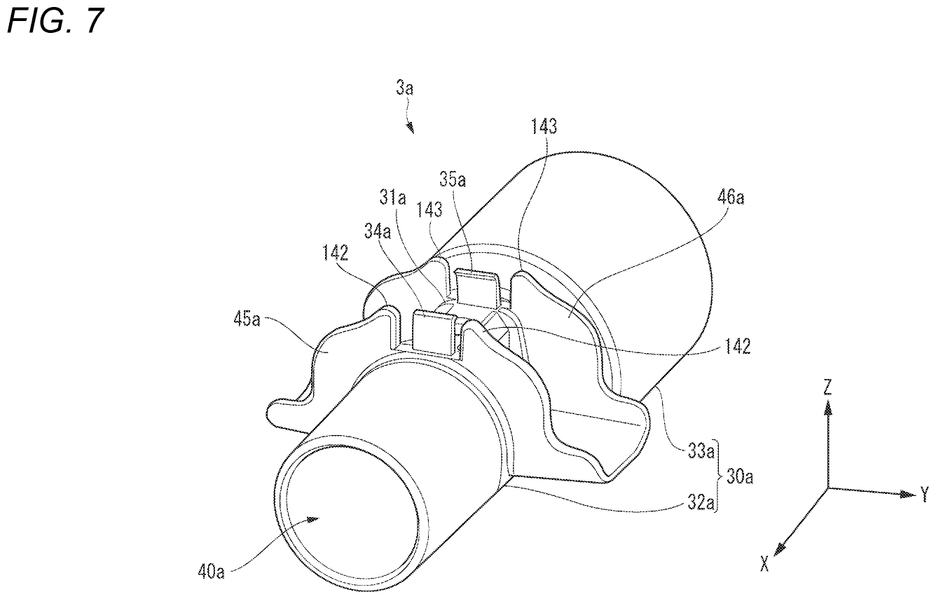

Next, an example of an incompatible airway adaptor will be described below with reference to FIGS. 6 and 7 to describe a difference between the compatible airway adaptor 3 according to the present embodiment and the incompatible airway adaptor. FIG. 6 is a perspective view of the respiratory gas sensor 2 and an incompatible airway adaptor 3a. FIG. 7 is a perspective view of the incompatible airway adaptor 3a.

Here, the incompatible airway adaptor is an airway adaptor that is not compatible with the respiratory gas sensor 2. That is, the respiratory gas sensor 2 is not attachable to the incompatible airway adaptor 3a. More specifically, the respiratory gas sensor 2 is not attachable to the incompatible airway adaptor 3a in a state in which the incompatible airway adaptor 3a and the respiratory gas sensor interfere with each other.

FIG. 6 illustrates a state in which a portion of the respiratory gas sensor 2 interferes with the incompatible airway adaptor 3a. In the following description, a respiratory gas sensor attachable to the incompatible airway adaptor 3a may be referred to as a respiratory gas sensor 2a. The respiratory gas sensor 2a is different from the respiratory gas sensor 2 according to the present embodiment in that no attachment preventing portion is provided.

As illustrated in FIGS. 6 and 7, the incompatible airway adaptor 3a is greatly different from the compatible airway adaptor 3 in that the first accommodation portions 36 and the second accommodation portions 37 are not provided. The incompatible airway adaptor 3a can include a tube portion 30a, a sensor attachment portion 31a, a first flange portion 45a, a second flange portion 46a, a first adapter-side engagement portion 34a, and a second adapter-side engagement portion 35a. The incompatible airway adaptor 3a can further include a pair of first interference protruding portions 142 and a pair of second interference protruding portions 143.

The tube portion 30a can include a respiratory gas ventilation passage 40a. The tube portion 30a can include a first tube portion 32a having a first outer diameter and a second tube portion 33a having a second outer diameter greater than the first outer diameter. The sensor attachment portion 31a determines a position of the respiratory gas sensor 2a relative to the incompatible airway adaptor 3a. Each of the first adapter engagement portion 34a and the second adapter engagement portion 35a engages with a corresponding portion of the respiratory gas sensor 2a.

The pair of first interference protruding portions 142 are configured such that the respiratory gas sensor 2 interferes with the incompatible airway adaptor 3a. The pair of first interference protruding portions 142 are protruding portions provided on the first flange portion 45a. As illustrated in FIG. 6, each of the pair of first interfering protruding portions 142 interferes with a corresponding one of the pair of first attachment preventing portions 22. In particular, each of the pair of first interference protruding portions 142 comes into contact with the corresponding first attachment preventing portion 22 such that the corresponding first attachment preventing portion 22 interferes with the incompatible airway adaptor 3a.

The pair of second interference protruding portions 143 are configured such that the respiratory gas sensor 2 interferes with the incompatible airway adaptor 3a. The pair of second interference protruding portions 143 are protruding portions provided on the second flange portion 46a. Each of the pair of second interference protruding portions 143 interferes with a corresponding one of the pair of second attachment preventing portions 25 (see FIG. 3). In particular, each of the pair of second interference protruding portions 143 comes into contact with the corresponding second attachment preventing portion 25 such that the corresponding second attachment preventing portion 25 interferes with the incompatible airway adaptor 3a. In this manner, the fitting portion 23 of the respiratory gas sensor 2 is not fitted to the sensor attachment portion 31a of the incompatible airway adaptor 3a, and thus the respiratory gas sensor 2 is not attachable to the incompatible airway adaptor 3a.

According to the present embodiment, the respiratory gas sensor 2 is attachable to the compatible airway adaptor 3 in a state in which the compatible airway adaptor 3 and the respiratory gas sensor 2 do not interfere with each other. On the other hand, the respiratory gas sensor 2 is not attachable to the incompatible airway adaptor 3a in a state in which the incompatible airway adaptor 3a that is not compatible with the respiratory gas sensor 2 and the respiratory gas sensor interfere with each other. In this manner, it is possible to provide the sensor attachment system 1 in which the respiratory gas sensor 2 is attachable or not attachable to an airway adaptor depending on a type of the airway adaptor, and it is possible to improve usability of the sensor attachment system 1 and the respiratory gas sensor 2.

For example, when the respiratory gas sensor 2 is a respiratory gas sensor that is applicable only to invasive positive pressure ventilation, the compatible airway adaptor 3 may be an intubation airway adaptor, while the incompatible airway adaptor 3a may be a non-intubation airway adaptor. In this case, the respiratory gas sensor 2 is attachable to the compatible airway adaptor 3 that is an intubation airway adaptor, and is not attachable to the incompatible airway adaptor 3a that is a non-intubation airway adaptor.

When the respiratory gas sensor 2 is a respiratory gas sensor for an adult, the compatible airway adaptor 3 may be an airway adaptor for an adult, while the incompatible airway adaptor 3a may be an airway adaptor for a neonate. In this case, the respiratory gas sensor 2 is attachable to the compatible airway adaptor 3 that is an airway adaptor for an adult, and is not attachable to the incompatible airway adaptor 3a that is an airway adaptor for a neonate.

In the present embodiment, it is possible to provide the sensor attachment system 1 in which the respiratory gas sensor 2 is attachable or not attachable to an airway adaptor depending on a type of the airway adaptor through the cooperation between the pair of first attachment preventing portions 22 and the pair of first accommodation portions 36 and the cooperation between the pair of second attachment preventing portions 25 and the pair of second accommodation portions 37. In this manner, the usability of the sensor attachment system 1 and the respiratory gas sensor 2 can be improved. In particular, the first attachment preventing portions 22 and the second attachment preventing portions 25 provided on the respiratory gas sensor 2 can reliably prevent the attachment of the respiratory gas sensor 2 to the incompatible airway adaptor 3a.

In the present embodiment, in the Y-axis direction, the pair of first attachment preventing portions 22 face each other through the first sensor-side engagement portion 21, and the pair of second attachment preventing portions 25 face each other through the second sensor-side engagement portion 26. According to such an arrangement structure, it is possible to reliably prevent the respiratory gas sensor 2 from being attached to the incompatible airway adaptor 3a, while it is possible to reliably attach the respiratory gas sensor 2 to the compatible airway adaptor 3. In this regard, the single first adapter-side engagement portion 34 is reliably snap-fitted to the first sensor-side engagement portion 21, and the single second adapter-side engagement portion 35 is reliably snap-fitted to the second sensor-side engagement portion 26. For this reason, the respiratory gas sensor 2 can be reliably attached to the compatible airway adaptor 3. On the other hand, since the pair of first attachment preventing portions 22 and the second attachment preventing portion 25 reliably interfere with the incompatible airway adaptor 3a, attachment between the respiratory gas sensor 2 and the incompatible airway adaptor 3a is reliably prevented.

In the present embodiment, the pair of first attachment preventing portions 22 and the pair of second attachment preventing portion 25 are columnar bodies extending in the Z-axis direction. For this reason, when the respiratory gas sensor 2 is inserted into the incompatible airway adaptor 3a along the Z-axis direction, the pair of the first attachment preventing portions 22 and the pair of second attachment preventing portions 25 extending in the Z-axis direction reliably interfere with the incompatible airway adaptor 3a. In particular, as illustrated in FIG. 6, before the first adapter-side engagement portion 34a is snap-fitted to the first sensor-side engagement portion 21, the pair of first attachment preventing portions 22 interfere with a portion of the incompatible airway adaptor 3a. in a same or similar manner, before the second adapter-side engagement portion 35a is reliably snap-fitted to the second sensor-side engagement portion 26, the pair of second attachment preventing portions 25 interfere with a part of the incompatible airway adaptor 3a. In this manner, a medical worker can reliably grasp that the respiratory gas sensor 2 is not attachable to the incompatible airway adaptor 3a. Further, since the first attachment preventing portions 22 and the second attachment preventing portions 25 are columnar bodies extending in the Z-axis direction, the rigidity of the first attachment preventing portions 22 and the second attachment preventing portions 25 in the Z-axis direction can be improved, and the first attachment preventing portions 22 and the second attachment preventing portions 25 can be suitably prevented from being damaged by an external force in the Z-axis direction.

Although the embodiment of the presently disclosed subject matter is described above, the technical scope of the presently disclosed subject matter should not be construed as being limited to the description of the embodiment. It is understood by those skilled in the art that the present embodiment is an example and various modifications may be made within the scope of the inventions described in the claims. The technical scope of the presently disclosed subject matter should be determined based on the scope of the inventions described in the claims and the scope of equivalents thereof.

In the present embodiment, the first attachment preventing portions 22 and the second attachment preventing portions 25 are provided on the respiratory gas sensor 2, and the number of attachment preventing portions is not particularly limited. In a same or similar manner, the number of the first accommodation portions 36 accommodating the first attachment preventing portions 22 and the number of the second accommodation portions 37 accommodating the second attachment preventing portions 25 are not particularly limited. For example, the single first attachment preventing portion 22 may be provided on the first surface 24 of the respiratory gas sensor 2, and the single second attachment preventing portion 25 may be provided on the second surface 27 of the respiratory gas sensor 2. In this case, the number of the first accommodation portions 36 and the number of the second accommodation portions 37 may be one.

In the present embodiment, the first attachment preventing portions 22 are disposed in the vicinity of the first sensor-side engagement portion 21, and the second attachment preventing portions 25 are disposed in the vicinity of the second sensor-side engagement portion 26.

In the present embodiment, attachment preventing portions are provided on the respiratory gas sensor 2 and accommodation portions that accommodate the attachment preventing portions are provided on the compatible airway adaptor 3, and the present embodiment is not limited thereto. In this regard, the attachment preventing portions may be provided on the airway adaptor, and the accommodation portions that accommodate the attachment preventing portions may be provided on the respiratory gas sensor. In this case, the attachment between the respiratory gas sensor and the airway adaptor is possible or not possible depending on a type of the respiratory gas sensor. In this case, each of plural attachment preventing portions provided on the airway adaptor may be a columnar body extending along the Z-axis direction. Further, each of plural accommodation portions provided on the respiratory gas sensor compatible with the airway adaptor (hereinafter referred to as compatible respiratory gas sensor) may be a groove extending along the Z-axis direction. In this way, by inserting each of the plural attachment preventing portions into the corresponding accommodation portion, the airway adaptor is attachable to the compatible respiratory gas sensor. On the other hand, a respiratory gas sensor that is not compatible with an airway adaptor (hereinafter referred to as incompatible respiratory gas sensor) is not provided with an accommodation portion that accommodates an attachment preventing portion. For this reason, when the plural attachment preventing portions provided on the airway adaptor interfere with the incompatible respiratory gas sensor, attachment between the airway adaptor and the incompatible respiratory gas sensor is reliably prevented.

The aforementioned embodiment is summarized as follows.

The present disclosure provides a sensor attachment system, a respiratory gas sensor, and a compatible airway adaptor with improved usability. In particular, the present disclosure provides a sensor attachment system in which a respiratory gas sensor is attachable or not attachable to an airway adaptor depending on a type of the airway adaptor.

A first aspect of the present disclosure relates to a sensor attachment system including: a sensor configured to detect a state of respiratory gas of a subject; and a compatible airway adaptor compatible with the sensor. The sensor includes a respiratory gas detector configured to detect the state of the respiratory gas of the subject, and a sensor-side engagement portion engageable with the compatible airway adaptor. The compatible airway adaptor includes a tube portion having a respiratory gas ventilation passage through which the respiratory gas passes, and an adapter-side engagement portion engageable with the sensor-side engagement portion. The sensor is attachable to the compatible airway adaptor in a state in which the compatible airway adaptor and the sensor do not interfere with each other, and the sensor is not attachable to an incompatible airway adaptor that is not compatible with the sensor in a state in which the incompatible airway adaptor and the sensor interfere with each other.

According to the above configuration, the respiratory gas sensor is attachable to the compatible airway adapter in a state in which the compatible air adapter that is compatible with the respiratory gas sensor and the respiratory gas sensor do not interfere with each other. On the other hand, the respiratory gas sensor is not attachable to the incompatible airway adaptor in a state in which the incompatible airway adaptor that is not compatible with the respiratory gas sensor and the respiratory gas sensor interfere with each other. In this manner, it is possible to provide a sensor attachment system in which a respiratory gas sensor is attachable or not attachable to an airway adaptor depending on a type of the airway adaptor, and it is possible to improve usability of the sensor attachment system.

A second aspect of the present disclosure relates to a respiratory gas sensor. The respiratory gas sensor is configured to detect a state of respiratory gas of a subject and is attachable to a compatible airway adaptor compatible with the respiratory gas sensor. The respiratory gas sensor includes: a respiratory gas detector configured to detect the state of the respiratory gas of the subject; and a sensor-side engagement portion engageable with the compatible airway adaptor. The respiratory gas sensor is attachable to the compatible airway adaptor in a state in which the compatible airway adaptor and the respiratory gas sensor do not interfere with each other, and the respiratory gas sensor is not attachable to an incompatible airway adaptor that is not compatible with the respiratory gas sensor in a state in which the incompatible airway adaptor and the respiratory gas sensor interfere with each other.

According to the above configuration, the respiratory gas sensor is attachable to the compatible airway adapter in a state in which the compatible air adapter that is compatible with the respiratory gas sensor and the respiratory gas sensor do not interfere with each other. On the other hand, the respiratory gas sensor is not attachable to the incompatible airway adaptor in a state in which the incompatible airway adaptor that is not compatible with the respiratory gas sensor and the respiratory gas sensor interfere with each other. In this manner, a respiratory gas sensor is attachable or not attachable to an airway adaptor depending on a type of the airway adaptor, and it is possible to improve usability of the respiratory gas sensor.

A third aspect of the present disclosure relates to a compatible airway adaptor. The compatible airway adaptor is attachable to a respiratory gas sensor configured to detect a state of respiratory gas of a subject. The compatible airway adaptor includes: a tube portion having a respiratory gas ventilation passage through which the respiratory gas passes; and an adapter-side engagement portion configured to engage with the respiratory gas sensor. The respiratory gas sensor includes an attachment preventing portion configured to cause the respiratory gas sensor to be unattachable to an incompatible airway adaptor that is not compatible with the respiratory gas sensor, and the compatible airway adaptor further includes an accommodation portion configured to accommodate the attachment preventing portion to prevent interference with the attachment preventing portion.

According to the above configuration, the accommodation portion configured to accommodate the attachment preventing portion is provided on the compatible airway adaptor to prevent interference with the attachment preventing portion provided on the respiratory gas sensor, and thus the respiratory gas sensor is attachable to the compatible airway adaptor. In this manner, it is possible to provide a compatible airway adaptor that is compatible with a respiratory gas sensor.

According to the present disclosure, it is possible to provide a sensor attachment system, a respiratory gas sensor, and a compatible airway adaptor with improved usability. In particular, it is possible to provide a sensor attachment system in which a respiratory gas sensor is attachable or not attachable to an airway adaptor depending on a type of the airway adaptor.

1. A sensor attachment system comprising:
a respiratory gas sensor configured to detect a state of respiratory gas of a subject; and
a compatible airway adaptor compatible with the respiratory gas sensor,
wherein the respiratory gas sensor includes
a respiratory gas detector configured to detect the state of the respiratory gas of the subject, and
a sensor-side engagement portion engageable with the compatible airway adaptor,
wherein the compatible airway adaptor includes
a tube portion having a respiratory gas ventilation passage through which the respiratory gas passes, and
an adapter-side engagement portion engageable with the sensor-side engagement portion,
wherein the respiratory gas sensor is attachable to the compatible airway adaptor in a state in which the compatible airway adaptor and the respiratory gas sensor do not interfere with each other, and
wherein the respiratory gas sensor is not attachable to an incompatible airway adaptor that is not compatible with the respiratory gas sensor in a state in which the incompatible airway adaptor and the respiratory gas sensor interfere with each other.

2. The sensor attachment system according to claim 1,
wherein the respiratory gas sensor further includes an attachment preventing portion provided on at least one of a first surface and a second surface opposite to the first surface of the respiratory gas sensor and configured to cause the respiratory gas sensor to be unattachable to the incompatible airway adaptor,
wherein the respiratory gas sensor is attachable to the compatible airway adaptor in a state in which the compatible airway adaptor and the attachment preventing portion do not interfere with each other, and
wherein the respiratory gas sensor is not attachable to the incompatible airway adaptor in a state in which the incompatible airway adaptor and the attachment preventing portion interfere with each other.

3. The sensor attachment system according to claim 2,
wherein the compatible airway adaptor further includes an accommodation portion configured to accommodate the attachment preventing portion to prevent the interference with the attachment preventing portion.

4. The sensor attachment system according to claim 2,
wherein the attachment preventing portion has a columnar body protruding from at least one of the first surface and the second surface.

5. The sensor attachment system according to claim 3,
wherein the respiratory gas sensor further includes a fitting portion configured to determine a position of the respiratory gas sensor relative to the compatible airway adaptor and to be fitted to a portion of the compatible airway adaptor,
wherein the compatible airway adaptor further includes a sensor attachment portion configured to determine the position of the respiratory gas sensor relative to the compatible airway adaptor and to be fitted to the fitting portion,
wherein the tube portion includes
a first tube portion having a first outer diameter, and
a second tube portion having a second outer diameter greater than the first outer diameter,
wherein the sensor attachment portion is between the first tube portion and the second tube portion in a longitudinal direction of the compatible airway adaptor, and
wherein the adapter-side engagement portion and the accommodation portion are between the first tube portion and the sensor attachment portion or between the second tube portion and the sensor attachment portion in the longitudinal direction.

6. The sensor attachment system according to claim 5,
wherein the sensor-side engagement portion includes
a first sensor-side engagement portion provided on the first surface, and
a second sensor-side engagement portion provided on the second surface,
wherein the adapter-side engagement portion includes
a first adapter-side engagement portion provided between the first tube portion and the sensor attachment portion in the longitudinal direction and engageable with the first sensor-side engagement portion, and
a second adapter-side engagement portion provided between the second tube portion and the sensor attachment portion in the longitudinal direction and configured to engage with the second sensor-side engagement portion,
wherein the attachment preventing portion includes
a first attachment preventing portion provided on the first surface, and
a second attachment preventing portion provided on the second surface, and
wherein the accommodation portion includes
a first accommodation portion provided between the first tube portion and the sensor attachment portion in the longitudinal direction and configured to accommodate the first attachment preventing portion to prevent the interference with the first attachment preventing portion, and
a second accommodation portion provided between the second tube portion and the sensor attachment portion in the longitudinal direction and configured to accommodate the second attachment preventing portion to prevent the interference with the second attachment preventing portion.

7. The sensor attachment system according to claim 6,
wherein the first attachment preventing portion includes a pair of the first attachment preventing portions facing each other through the first sensor-side engagement portion,
wherein the second attachment preventing portion includes a pair of the second attachment preventing portions facing each other through the second sensor-side engagement portion,
wherein the first accommodation portion includes a pair of the first accommodation portions facing each other through the first adapter-side engagement portion,
wherein the second accommodation portion includes a pair of second accommodation portions facing each other through the second adapter-side engagement portion,
wherein each of the pair of the first accommodation portions accommodates a corresponding one of the pair of the first attachment preventing portions to prevent interference with the corresponding first attachment preventing portion, and
wherein each of the pair of the second accommodation portions accommodates a corresponding one of the pair of the second attachment preventing portions to prevent interference with the corresponding second attachment preventing portion.

8. A respiratory gas sensor, wherein
the respiratory gas sensor is configured to detect a state of respiratory gas of a subject and is attachable to a compatible airway adaptor compatible with the respiratory gas sensor,
the respiratory gas sensor comprises:
a respiratory gas detector configured to detect the state of the respiratory gas of the subject; and
a sensor-side engagement portion engageable with the compatible airway adaptor,
wherein the respiratory gas sensor is attachable to the compatible airway adaptor in a state in which the compatible airway adaptor and the respiratory gas sensor do not interfere with each other, and
wherein the respiratory gas sensor is not attachable to an incompatible airway adaptor that is not compatible with the respiratory gas sensor in a state in which the incompatible airway adaptor and the respiratory gas sensor interfere with each other.

9. The respiratory gas sensor according to claim 8, further comprising:
an attachment preventing portion provided on at least one of a first surface and a second surface opposite to the first surface of the respiratory gas sensor and configured to cause the respiratory gas sensor to be unattachable to the incompatible airway adaptor,
wherein the respiratory gas sensor is attachable to the compatible airway adaptor in a state in which the compatible airway adaptor and the attachment preventing portion do not interfere with each other, and
wherein the respiratory gas sensor is not attachable to the incompatible airway adaptor in a state in which the incompatible airway adaptor and the attachment preventing portion interfere with each other.

10. A compatible airway adaptor, wherein
the compatible airway adaptor is attachable to a respiratory gas sensor configured to detect a state of respiratory gas of a subject,
the compatible airway adaptor comprises:
a tube portion having a respiratory gas ventilation passage through which the respiratory gas passes; and
an adapter-side engagement portion configured to engage with the respiratory gas sensor,
wherein the respiratory gas sensor includes an attachment preventing portion configured to cause the respiratory gas sensor to be unattachable to an incompatible airway adaptor that is not compatible with the respiratory gas sensor, and
wherein the compatible airway adaptor further includes an accommodation portion configured to accommodate the attachment preventing portion to prevent interference with the attachment preventing portion.
